(19) United States
(12) Patent Application Publication
(10) Pub. No.: US 2015/0376085 A1
(43) Pub. Date: Dec. 31, 2015

(54) ANTHRACENE COMPOUND, METHOD FOR PREPARING THE SAME, USE THEREOF AND ORGANIC LIGHT EMITTING DEVICE

(71) Applicants: BOE TECHNOLOGY GROUP CO., LTD., Beijing (CN); JILIN OLED MATERIAL TECH CO., LTD., Changchun City, Jilin Province (CN)

(72) Inventors: Yansong LI, Beijing (CN); Xiaoyu MA, Beijing (CN); Hui WANG, Beijing (CN); Na LI, Beijing (CN)

(73) Assignees: BOE TECHNOLOGY GROUP CO., LTD., Beijing (CN); JILIN OLED MATERIAL TECH CO., LTD., Changchun City, Jilin Province (CN)

(21) Appl. No.: 14/416,794

(22) PCT Filed: May 29, 2014
(86) PCT No.: PCT/CN2014/078780

§ 371 Date: Jan. 23, 2015

(30) Foreign Application Priority Data
Dec. 10, 2013 (CN) 201310666464.1

(51) Int. Cl. C07C 2/86; C07D 213/06; C09K 11/02; C07D 213/127; C07C 15/20; C07C 13/58; C07D 215/04; C07D 277/66

(52) U.S. Cl. CPC C07C 2/861; C07D 215/04; C07D 213/06; C07D 277/66; C07D 213/127; C07C 15/20; C07C 13/58; C09K 11/025; H01L 51/5012

(57) ABSTRACT

Provided are anthracene compound, method for preparing the same and use thereof, as well as organic electroluminescent device containing the same. The anthracene compound represented by a formula:

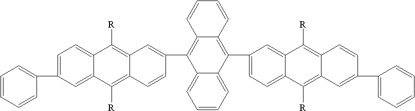

wherein R is selected from C6-C19 aryl, fused ring aryl, or substituted or unsubstituted heterocyclic aryl.

### CROSS-REFERENCE TO RELATED APPLICATION

This application is the U.S. national phase of PCT Application No. PCT/CN2014/078780 filed on May 29, 2014, which claims priority to Chinese Patent Application No. 201310666464.1 filed on Dec. 10, 2013, the disclosures of which are incorporated in their entirety by reference herein.

### TECHNICAL FIELD

The present disclosure relates to the field of an organic optoelectronic material, particularly relates to an anthracene compound, a method for preparing the same and a use thereof.

### BACKGROUND

An organic electroluminescent device generally consists of a pair of opposing electrodes, i.e., a cathode and an anode, and a layer containing an organic material. When a voltage is applied between the anode and the cathode, a hole is injected into a light emitting layer from the anode through a hole transport layer. At the same time, an electron is injected into the light emitting layer from the cathode through an electron transport layer. In a region of the light emitting layer, carriers rearranged to form excitons. Molecules of the light emitting layer emit light caused by the excitons changing from an excited state into a ground state, thereby a light emitting phenomenon occurs. The light emitting materials are divided into two groups according to a light emitting mechanism: one group is formed by a fluorescent material using singlet excitons; the other group is formed by a phosphorescent substance using triplet excitons.

An organic electroluminescence phenomenon is a phenomenon caused by currents passing through an interior of a specific organic molecule and then converting into visible lights. The organic electroluminescent device generally includes an anode, a cathode, and the layer of the organic material between the anode and the cathode. In this regard, the layer of the organic material may include a multilayer structure formed of different materials for improving efficiency and stability of manufactured organic electroluminescent device. For example, the layer of the organic material layer may include a hole injection layer, a hole transport layer, a light emitting layer, an electron transport layer, an electron injection layer and on the like.

The light emitting material can emit lights showing colors such as blue, green, red and yellow. For improving the light emitting efficiency of the light emitting layer, other light emitting materials having a higher quantum yield may be added in the light emitting layer. It is known that the excitons have a tendency of transferring their energy to materials with a smaller band gap of a recombined portion located nearby. Thus, a dopant is selected from a material having a higher quantum yield and a smaller band gap (larger wavelength) comparing with a host material; otherwise, energy of the excitons transfers to a host material having a lower quantum yield, and therefore a weak emission or even a non-emission occurs.

In order to effectively exert excellent characteristics of the organic electroluminescent device, a stable and efficient material may be used for the layer of the organic materials in the organic electroluminescent device, i.e., the hole injection layer, the hole transport layer, the light emitting layer, the electron transport layer, the electron injection layer and the like. But so far there is no ideal, stable and efficient organic light emitting material for the layer of the organic material. Therefore, study on new materials is imperative.

### SUMMARY

In view of the above-described problems, in the present disclosure anthracene is introduced with an aromatic group having a high efficiency and a good thermal stability, which improves light emitting efficiency and stability. An object of the present disclosure is to provide an anthracene compound, a method for preparing the same and a use thereof, directing to proving a novel and efficient organic electroluminescent material.

Technical solutions of the present disclosure are shown as below:

An anthracene compound represented by a formula:

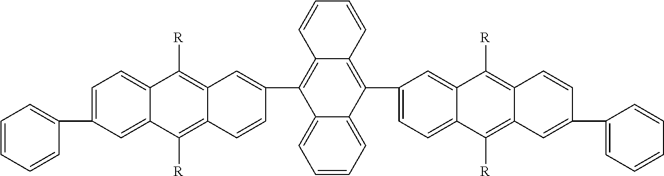

wherein R is selected from C6-C19 aryl, fused ring aryl, or substituted or unsubstituted heterocyclic aryl.

Specifically, R is selected from C6-050 phenyl, biphenyl, naphthyl, quinolyl, phenanthryl, pyridyl, phenalenyl, 9,9-dimethyl-fluorenyl, terphenyl, anthryl, aromatic azyl, carbazolyl, benzothiazolyl, thienyl, aromatic azyl, substituted or unsubstituted heterocyclic aryl, or anilino.

Specifically, the anthracene compound is represented by any one of following formulas:

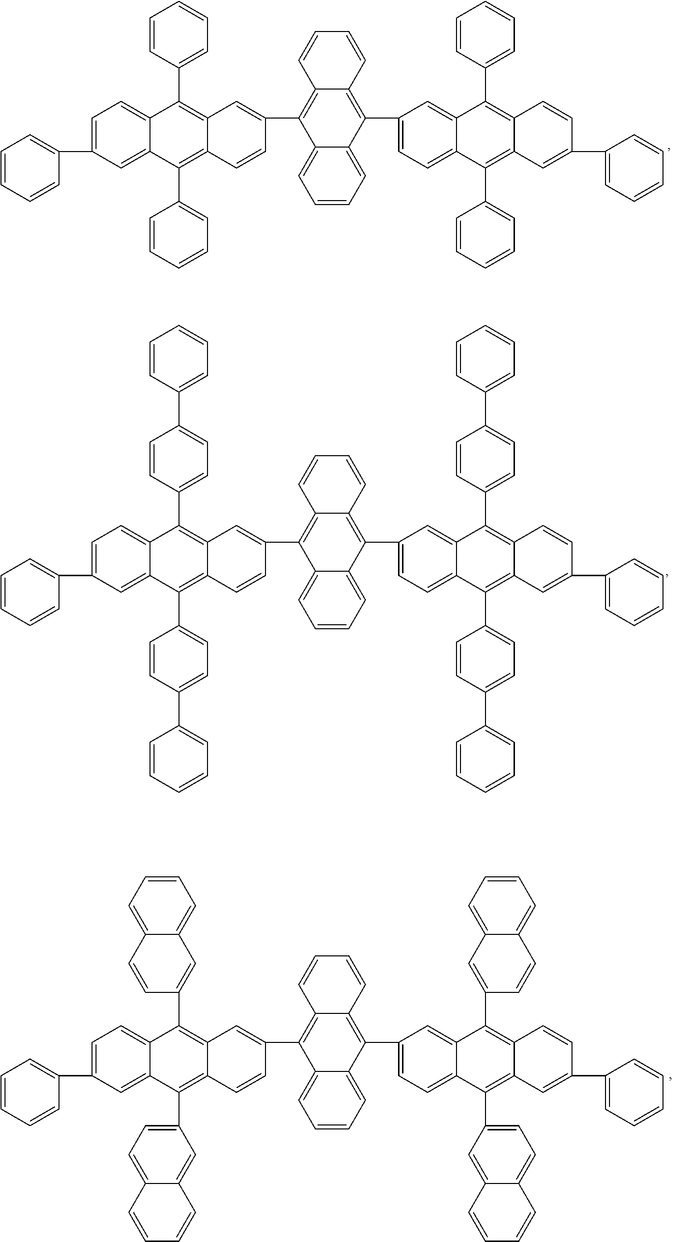

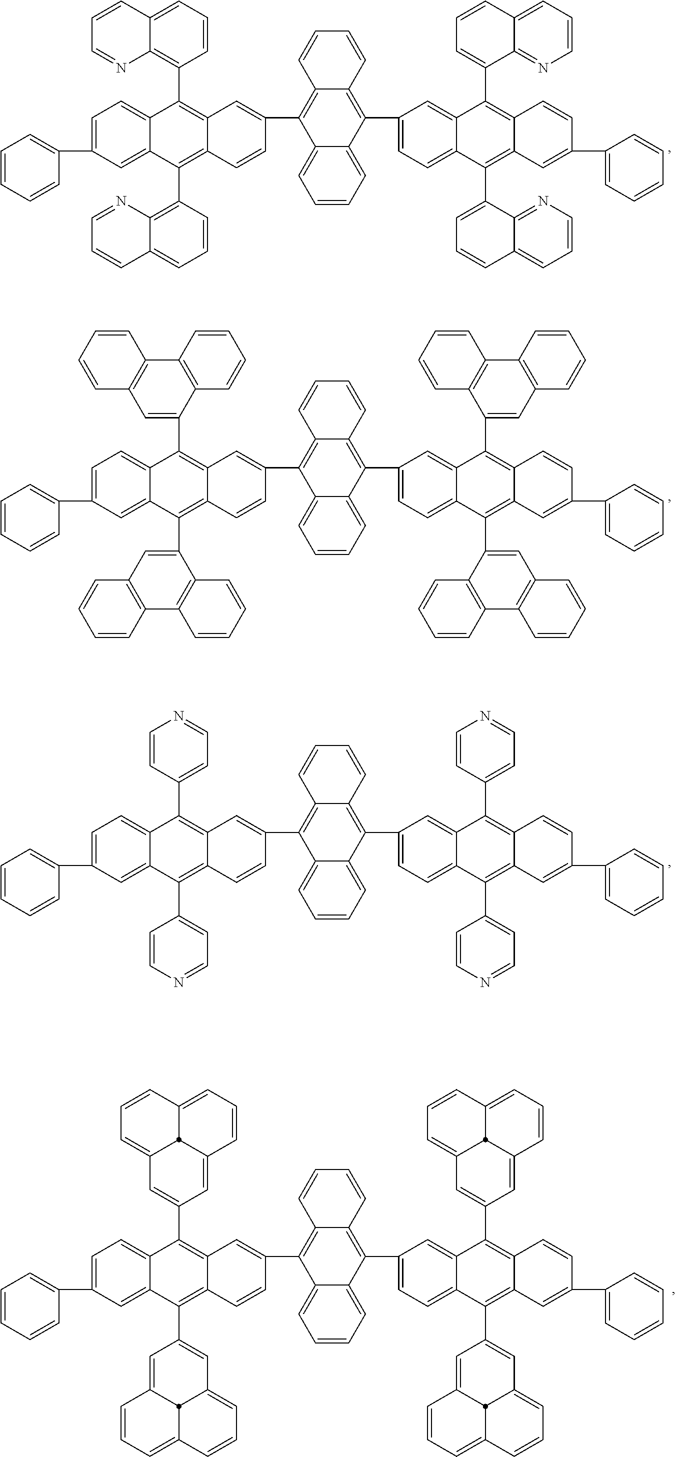

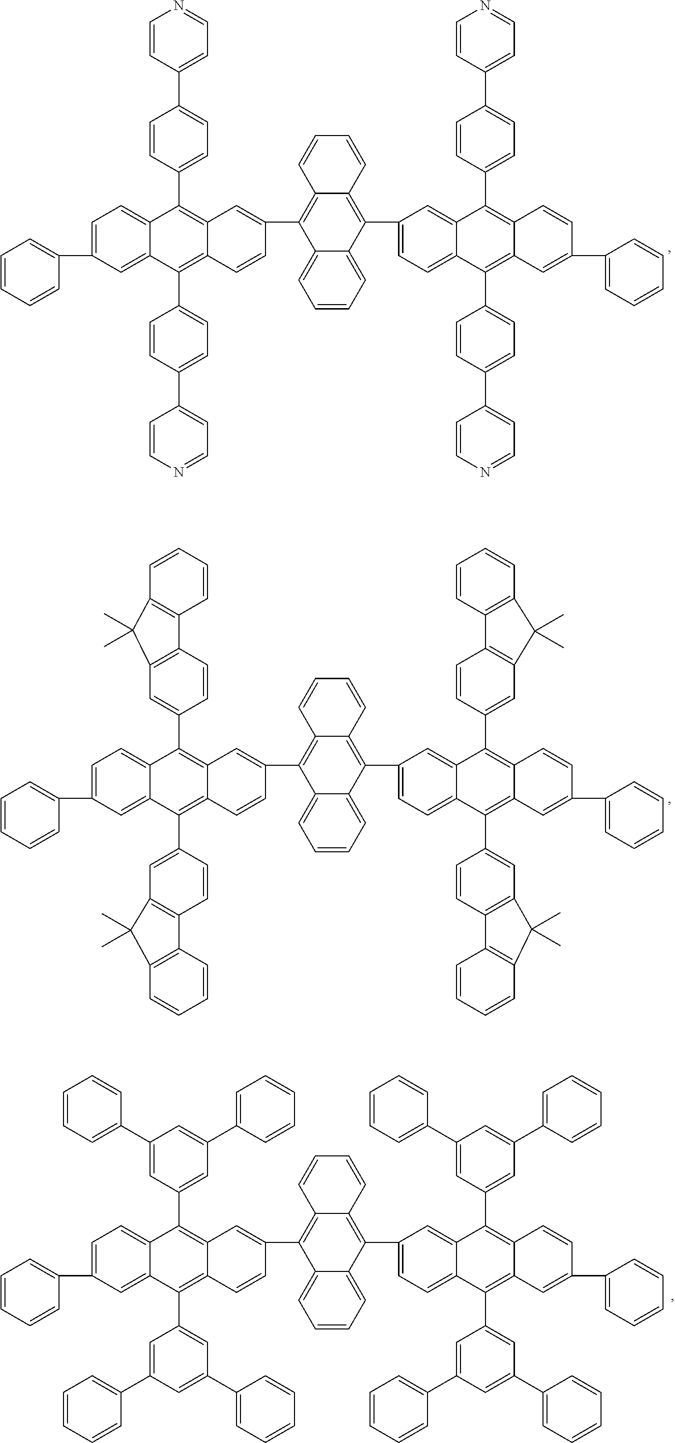

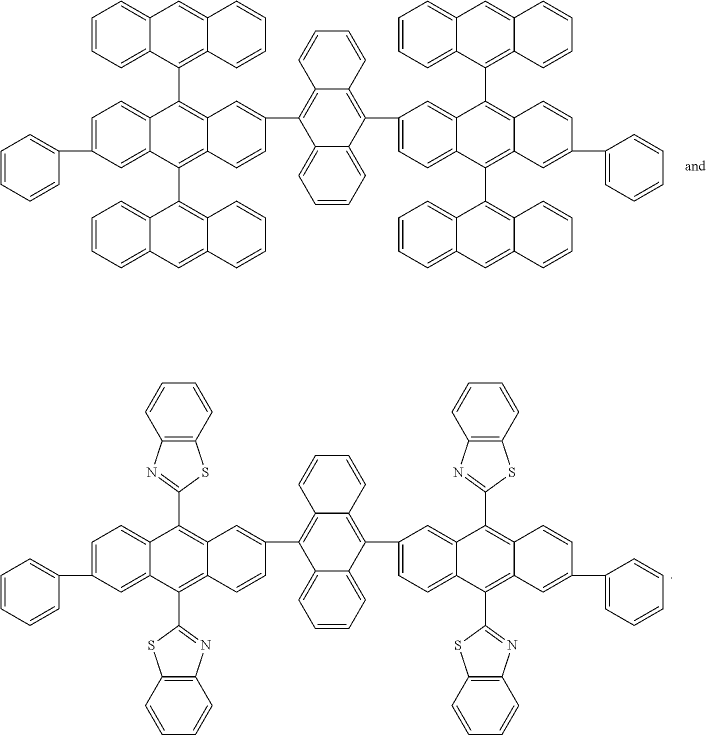

The present disclosure also provides a method for preparing the above anthracene compound, which includes the following steps:

step S1: degassing a reaction vessel, and adding

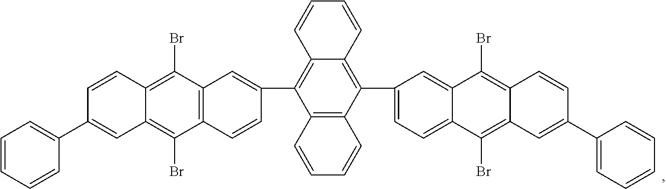 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]

R-boric acid, potassium carbonate and methylbenzene thereinto;

step S2: adding a catalyst, increasing a temperature of the reaction vessel to 70° C. and refluxing for reacting sufficiently; and

step S3: extracting, washing, drying and purifying by column chromatography, to obtain the anthracene compound,

wherein R is selected from C6-C19 aryl, fused ring aryl, or substituted or unsubstituted heterocyclic aryl.

Specifically, in the step S1,

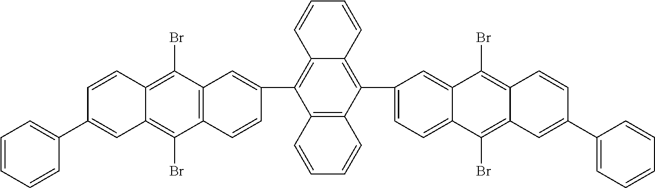 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]

is obtained from 9,10-anthraquinone.

Specifically, in the step S1,

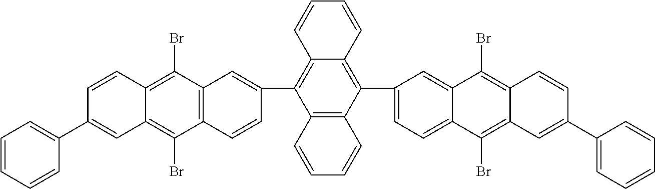 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]

is obtained by a multi-step reaction of alcoholization, dehydration and bromination using 2-bromo-6-benzanthracene and 9,10-anthraquinone as raw materials, which includes the following steps:

step N1: degassing a reaction vessel, and adding 2-bromo-6-benzanthracene and tetrahydrofuran thereinto;

step N2: decreasing a temperature of a reaction system, and adding n-BuLi;

step N3: adding 9,10-anthraquinone;

step N4: increasing the temperature of the reaction system to a room temperature, and adding NH₄Cl to stop the reaction after reacting sufficiently;

step N5: extracting, washing, drying and purifying by column chromatography to obtain

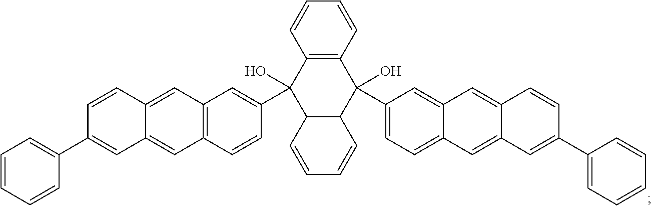 [SMILES: OC1(c2ccc3cc4cc(-c5ccccc5)ccc4cc3c2)c2ccccc2C(O)(c2ccc3cc4cc(-c5ccccc5)ccc4cc3c2)C2C=CC=CC21]

step N6: adding potassium iodide, sodium dihydrogen phosphate and acetic acid therein to obtain

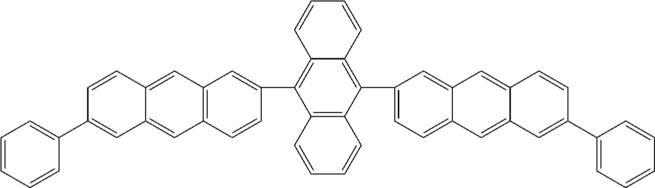 [SMILES: c1ccc(-c2ccc3cc4cc(-c5c6ccccc6c(-c6ccc7cc8cc(-c9ccccc9)ccc8cc7c6)c6ccccc56)ccc4cc3c2)cc1]

by a dehydration reaction; and

step N7: brominating by adding bromine water to obtain

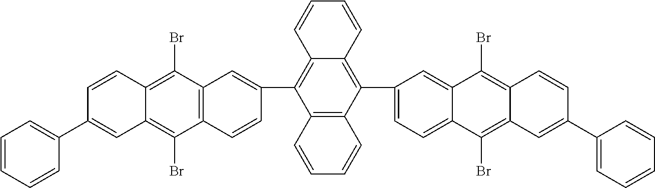 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]

in the step S1.

Specifically, the R-boric acid is selected from phenylboric acid, 4-biphenylboric acid, 2-naphthylboric acid, 8-quinolylboric acid, 9-phenanthrylboric acid, 4-pyridylboric acid, phenalenylboric acid, 4-(4-pyridyl)-phenylboric acid, 9,9-dimethyl-fluorenylboric acid, 3,5-diphenyl-phenylboric acid, 9-anthraceneboric acid, or 2-benzothiazolylboric acid.

The anthracene compound according to the present disclosure may be used as a fluoresce host material, a hole injection material or a hole transport material in the organic electroluminescent device.

Specifically, the anthracene compound is used as a fluorescent green host material in the organic electroluminescent device.

The anthracene compound according to the present disclosure may be used for manufacturing an organic electroluminescent device. The organic electroluminescent device may include a first electrode, a second electrode; and one or more organic compound layers between the first electrode and the second electrode, wherein at least one organic compound layer includes the anthracene compound.

The anthracene compound according to the present disclosure has a high light emitting efficiency, indicating that such compound may be used as a light emitting material or a light emitting host material; particularly the compound may be used as a fluorescence host material. The anthracene compound also has a high glass transition temperature, being difficult to be crystallized, and may be used in the organic electroluminescent device, showing a higher efficiency, a higher brightness, a longer product life and a better charge transportability, so that the organic electroluminescent device may have a prolonged life and a decreased production cost.

### DETAILED DESCRIPTION

The present disclosure provides an anthracene compound, a method for preparing the same and a use thereof. To make the objects, the technical solutions and the advantages of the present disclosure clearer and more apparent, detailed descriptions are made below. It should be understood that specific examples described hereinafter are only used to explain the present disclosure, but not intended to limit the present disclosure.

The present disclosure provides an anthracene compound represented by a formula:

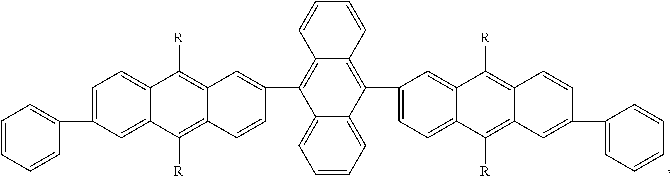

in which R is selected from C6-C19 aryl, fused ring aryl, or substituted or unsubstituted heterocyclic aryl.

Specifically, R is selected from C6-C50 phenyl, biphenyl, naphthyl, quinolyl, phenanthryl, pyridyl, phenalenyl, 9,9-dimethyl-fluorenyl, terphenyl, anthryl, aromatic azyl, carbazolyl, benzothiazolyl, thienyl, aromatic azyl, substituted or unsubstituted heterocyclic aryl, or anilino.

More specifically, the anthracene compound is represented by any one of formulas 1 to 12 in Table 1.

TABLE 1

| | |
|---|---|
| 1 | 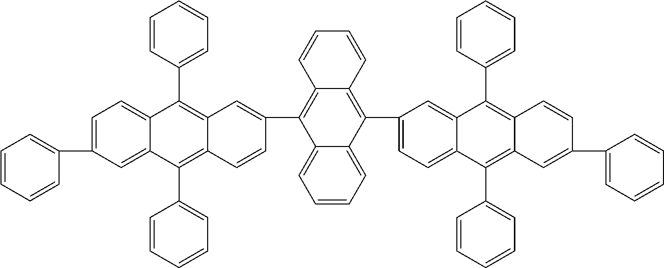 [SMILES: c1ccc(-c2ccc3c(-c4ccccc4)c4cc(-c5c6ccccc6c(-c6ccc7c(-c8ccccc8)c8cc(-c9ccccc9)ccc8c(-c8ccccc8)c7c6)c6ccccc56)ccc4c(-c4ccccc4)c3c2)cc1] |
| 2 | 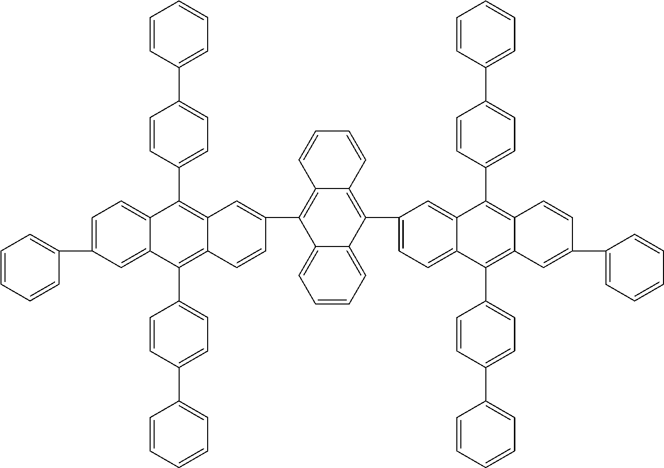 [SMILES: c1ccc(-c2ccc(-c3c4ccc(-c5c6ccccc6c(-c6ccc7c(-c8ccc(-c9ccccc9)cc8)c8cc(-c9ccccc9)ccc8c(-c8ccc(-c9ccccc9)cc8)c7c6)c6ccccc56)cc4c(-c4ccc(-c5ccccc5)cc4)c4ccc(-c5ccccc5)cc34)cc2)cc1] |
| 3 | 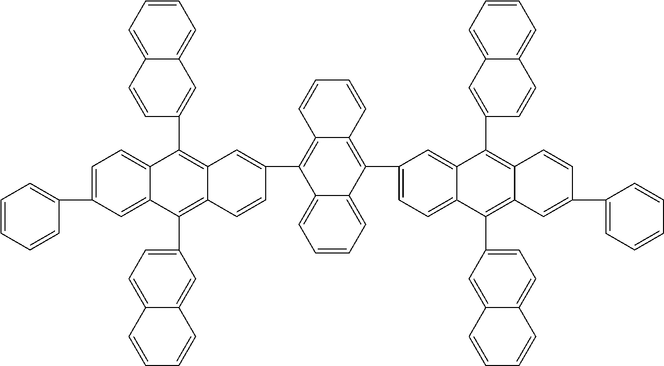 [SMILES: c1ccc(-c2ccc3c(-c4ccc5ccccc5c4)c4cc(-c5c6ccccc6c(-c6ccc7c(-c8ccc9ccccc9c8)c8cc(-c9ccccc9)ccc8c(-c8ccc9ccccc9c8)c7c6)c6ccccc56)ccc4c(-c4ccc5ccccc5c4)c3c2)cc1] |
| 4 | 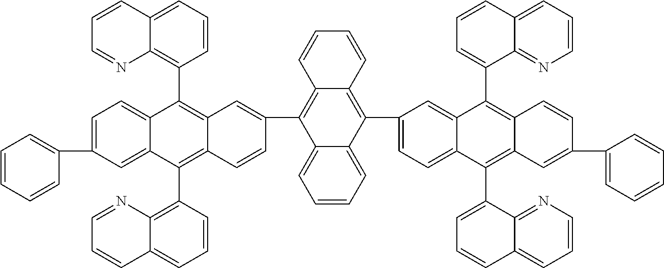 [SMILES: c1ccc(-c2ccc3c(-c4cccc5cccnc45)c4cc(-c5c6ccccc6c(-c6ccc7c(-c8cccc9cccnc89)c8cc(-c9ccccc9)ccc8c(-c8cccc9cccnc89)c7c6)c6ccccc56)ccc4c(-c4cccc5cccnc45)c3c2)cc1] |
| 5 | 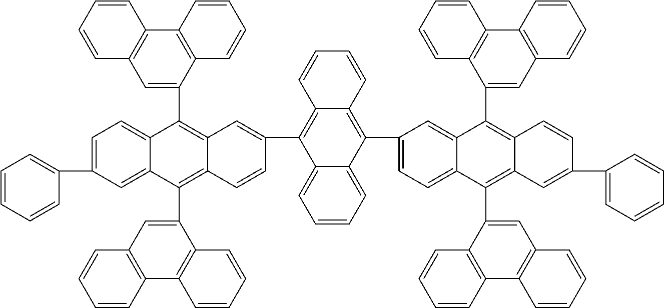 [SMILES: c1ccc(-c2ccc3c(-c4cc5ccccc5c5ccccc45)c4cc(-c5c6ccccc6c(-c6ccc7c(-c8cc9ccccc9c9ccccc89)c8cc(-c9ccccc9)ccc8c(-c8cc9ccccc9c9ccccc89)c7c6)c6ccccc56)ccc4c(-c4cc5ccccc5c5ccccc45)c3c2)cc1] |
| 6 | 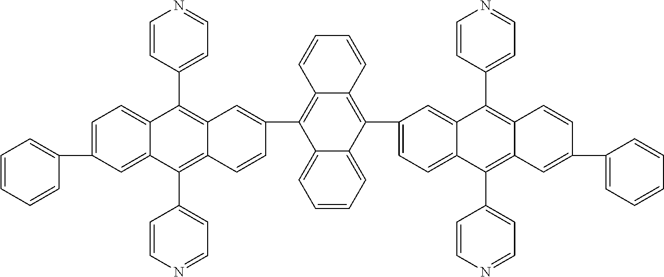 [SMILES: c1ccc(-c2ccc3c(-c4ccncc4)c4cc(-c5c6ccccc6c(-c6ccc7c(-c8ccncc8)c8cc(-c9ccccc9)ccc8c(-c8ccncc8)c7c6)c6ccccc56)ccc4c(-c4ccncc4)c3c2)cc1] |
| 7 | 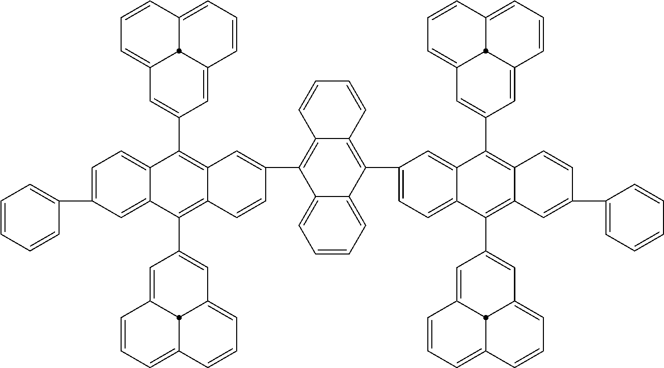 |
| 8 | 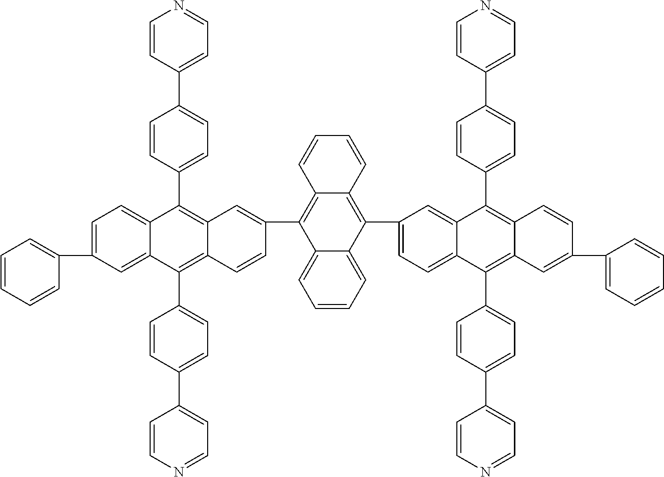 [SMILES: c1ccc(-c2ccc3c(-c4ccc(-c5ccncc5)cc4)c4cc(-c5c6ccccc6c(-c6ccc7c(-c8ccc(-c9ccncc9)cc8)c8cc(-c9ccccc9)ccc8c(-c8ccc(-c9ccncc9)cc8)c7c6)c6ccccc56)ccc4c(-c4ccc(-c5ccncc5)cc4)c3c2)cc1] |
| 9 | 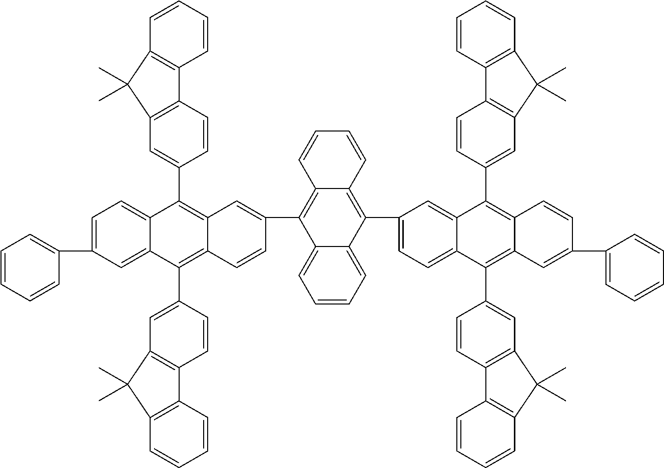 [SMILES: CC1(C)c2ccccc2-c2ccc(-c3c4ccc(-c5c6ccccc6c(-c6ccc7c(-c8ccc9c(c8)C(C)(C)c8ccccc8-9)c8cc(-c9ccccc9)ccc8c(-c8ccc9c(c8)C(C)(C)c8ccccc8-9)c7c6)c6ccccc56)cc4c(-c4ccc5c(c4)C(C)(C)c4ccccc4-5)c4ccc(-c5ccccc5)cc34)cc21] |
| 10 | 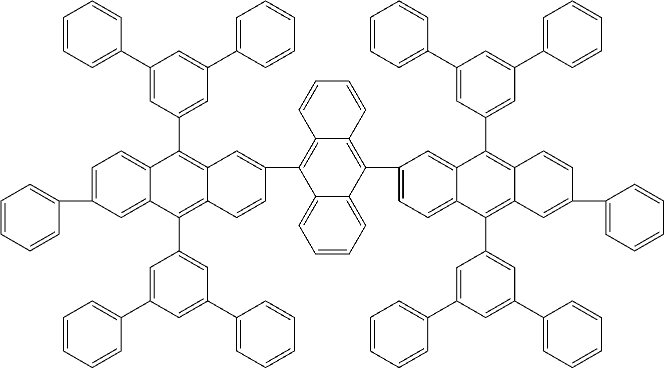 [SMILES: c1ccc(-c2cc(-c3ccccc3)cc(-c3c4ccc(-c5c6ccccc6c(-c6ccc7c(-c8cc(-c9ccccc9)cc(-c9ccccc9)c8)c8cc(-c9ccccc9)ccc8c(-c8cc(-c9ccccc9)cc(-c9ccccc9)c8)c7c6)c6ccccc56)cc4c(-c4cc(-c5ccccc5)cc(-c5ccccc5)c4)c4ccc(-c5ccccc5)cc34)c2)cc1] |
| 11 | 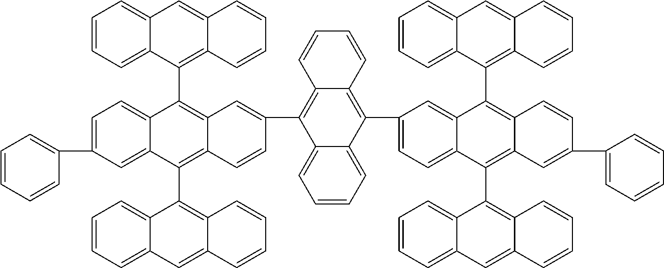 [SMILES: c1ccc(-c2ccc3c(-c4c5ccccc5cc5ccccc45)c4cc(-c5c6ccccc6c(-c6ccc7c(-c8c9ccccc9cc9ccccc89)c8cc(-c9ccccc9)ccc8c(-c8c9ccccc9cc9ccccc89)c7c6)c6ccccc56)ccc4c(-c4c5ccccc5cc5ccccc45)c3c2)cc1] |
| 12 | 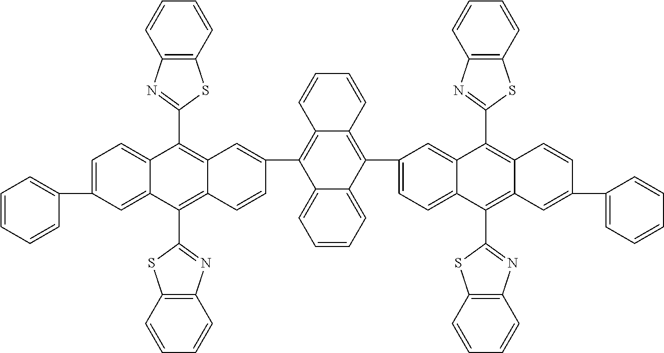 [SMILES: c1ccc(-c2ccc3c(-c4nc5ccccc5s4)c4cc(-c5c6ccccc6c(-c6ccc7c(-c8nc9ccccc9s8)c8cc(-c9ccccc9)ccc8c(-c8nc9ccccc9s8)c7c6)c6ccccc56)ccc4c(-c4nc5ccccc5s4)c3c2)cc1] |

The present disclosure also provides a method for preparing the above anthracene compound, including the following steps:

step S1: degassing a reaction vessel, and adding

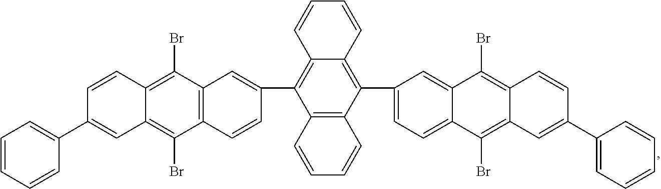 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]

R-boric acid, potassium carbonate and methylbenzene thereinto;

step S2: adding a catalyst, increasing a temperature of the reaction vessel to 70° C. and refluxing for reacting sufficiently; and

step S3: extracting, washing, drying and purifying by column chromatography, to obtain the anthracene compound,

in which R is selected from C6-C19 aryl, fused ring aryl, or substituted or unsubstituted heterocyclic aryl.

Specifically, in the step S1,

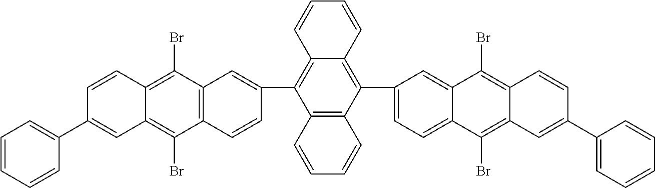 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]

is obtained from 9,10-anthraquinone.

Specifically, in the step S1,

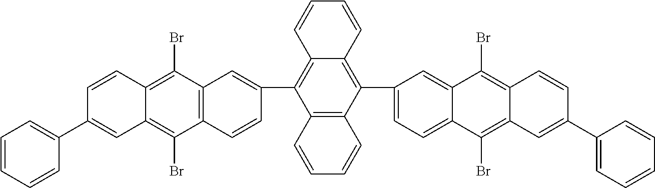 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]

is obtained by a multi-step reaction of alcoholization, dehydration and bromination using 2-bromo-6-benzanthracene and 9,10-anthraquinone as raw materials, which includes the following steps:

step N1: degassing a reaction vessel, and adding 2-bromo-6-benzanthracene and tetrahydrofuran thereinto;

step N2: decreasing a temperature of a reaction system, and adding n-BuLi; step N3: adding 9,10-anthraquinone;

step N4: increasing the temperature of the reaction system to a room temperature, and adding NH₄Cl to stop the reaction after reacting sufficiently;

step N5: extracting, washing, drying, and purifying by column chromatography to obtain

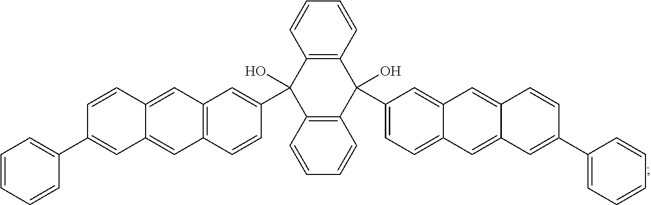 [SMILES: OC1(c2ccc3cc4cc(-c5ccccc5)ccc4cc3c2)c2ccccc2C(O)(c2ccc3cc4cc(-c5ccccc5)ccc4cc3c2)c2ccccc21]

step N6: adding potassium iodide, sodium dihydrogen phosphate and acetic acid to obtain

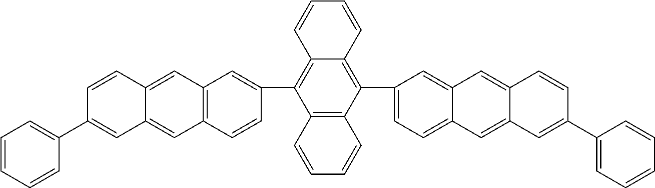 [SMILES: c1ccc(-c2ccc3cc4cc(-c5c6ccccc6c(-c6ccc7cc8cc(-c9ccccc9)ccc8cc7c6)c6ccccc56)ccc4cc3c2)cc1]

by a dehydration reaction; and

step N7: brominating by adding bromine water to obtain

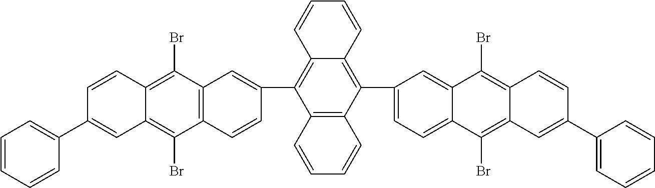 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]

in the step S1.

Specifically, R-boric acid is selected from phenylboric acid, 4-biphenylboric acid, 2-naphthylboric acid, 8-quinolylboric acid, 9-phenanthrylboric acid, 4-pyridylboric acid, phenalenylboric acid, 4-(4-pyridyl)-phenylboric acid, 9,9-dimethyl-fluorenylboric acid, 3,5-diphenyl-phenylboric acid, 9-anthrylboric acid, or 2-benzothiazolylboric acid.

Specifically, in order to describe the method for preparing the anthracene compound according to the present disclosure in further details, the anthracene compounds 1, 8, and 11 in Table 1 are taken as examples for description.

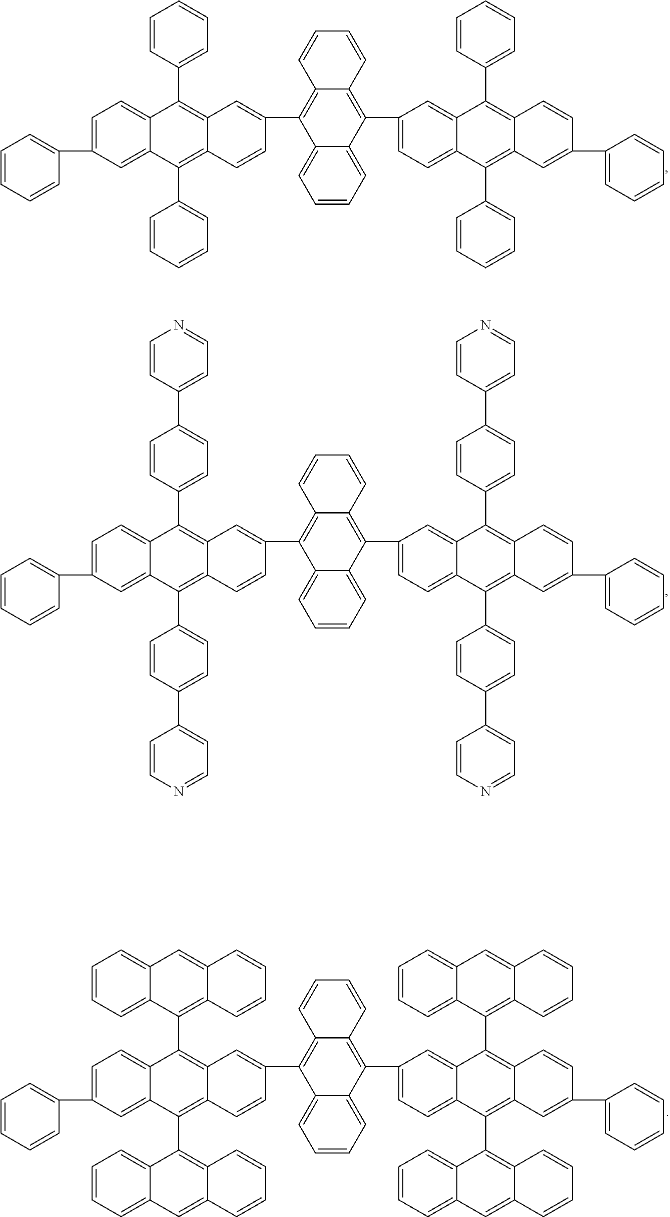

A specified reaction equation is shown as below:

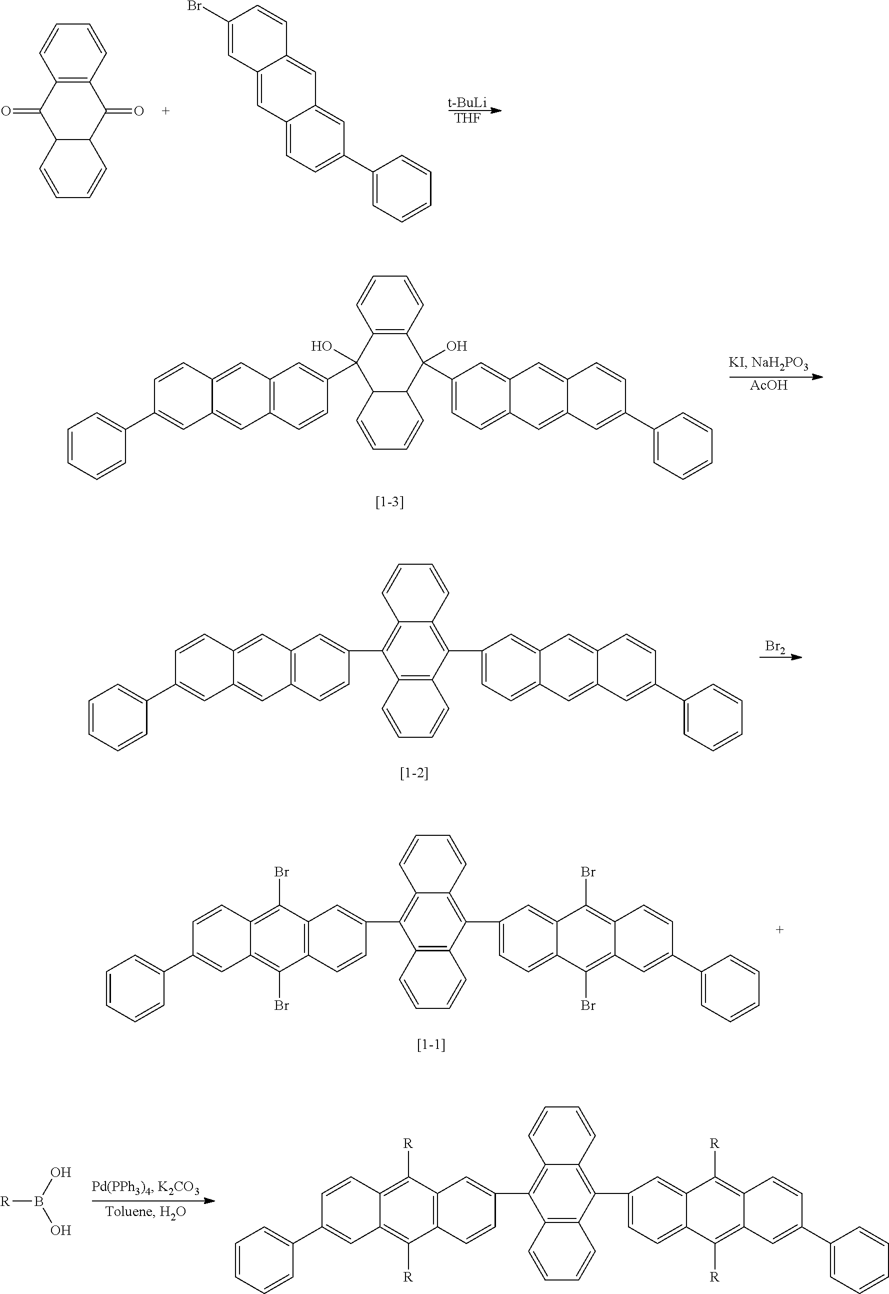

A method for synthesizing compound [1-1]:

1. 2-bromo-6-benzanthracene (83.31 g, 0.25 mol) and 250 mL of THF (tetrahydrofuran) were added into a reaction vessel under a protection of nitrogen atmosphere, and stirred for 10 min at a room temperature.

2. After the raw materials were completely dissolved, the temperature of the reaction system was decreased to −72° C., and then 100 mL of n-BuLi was slowly added.

3. After 3 hours reaction under the low temperature, 150 mL of THF solution containing 9,10-anthraquinone (21.02 g, 0.1 mol) was added thereinto.

4. The temperature of the reaction system was increased to the room temperature slowly, stirring for 24 hours. 500 mL of distilled water, 500 mL of supersaturated NH₄Cl solution and 500 mL of dichloromethane were added thereinto and stirred for 2 hours.

5. An organic layer was extracted, and dried under a vacuum atmosphere. The obtained solid was added with 1 L of acetone and stirred for 1 hour. After filtered and concentrated the organic solvent under a vacuum atmosphere, 48.89 g of compound [1-3] was obtained, being as a brownish solid with a yield of 68%.

6. Compound [1-3], potassium iodide (11.62 g, 0.07 mol), sodium dihydrogen phosphate (16.80 g, 0.14 mol), and 200 mL of acetic acid were added into a reaction vessel, and refluxed for 20 hours reaction with stirring. The temperature of the reaction system was cooled down to the room temperature after the reaction was completed, and then 500 mL of distilled water was added thereinto. After stirred, obtained solution was filtered under a vacuum atmosphere. The obtained solid was added with 500 mL of supersaturated NaHCO₃ solution, which was then stirred for 30 min and filtered under a vacuum atmosphere. Further, accordingly obtained solid was washed by 500 mL of supersaturated NaCl solution and 1 L of distilled water respectively, which was then filtered under a vacuum atmosphere. Then a deep yellow solid was obtained, which was dried in a vacuum atmosphere at the room temperature, and subsequently 40.63 g of compound was obtained with a yield of 85%.

7. Compound [1-2] (40.63 g, 0.06 mol) was added into a flask having a volume of 2 L under a protection of N₂ atmosphere, and 1 L of carbon tetrachloride was added thereinto as a solvent, which were reacted with bromine for 30 min at the room temperature. After the reaction was completed, which was confirmed by a thin layer chromatography (TLC) method, the reaction system was filtrated under a vacuum atmosphere, and stirred using 500 mL of acetone. After filtered again under a vacuum atmosphere, 49.72 g of target compound [1-1] was obtained, being as a light green solid with a yield of 83%.

Specifically, a method for synthesizing the compound represented by formula 1 is shown as below:

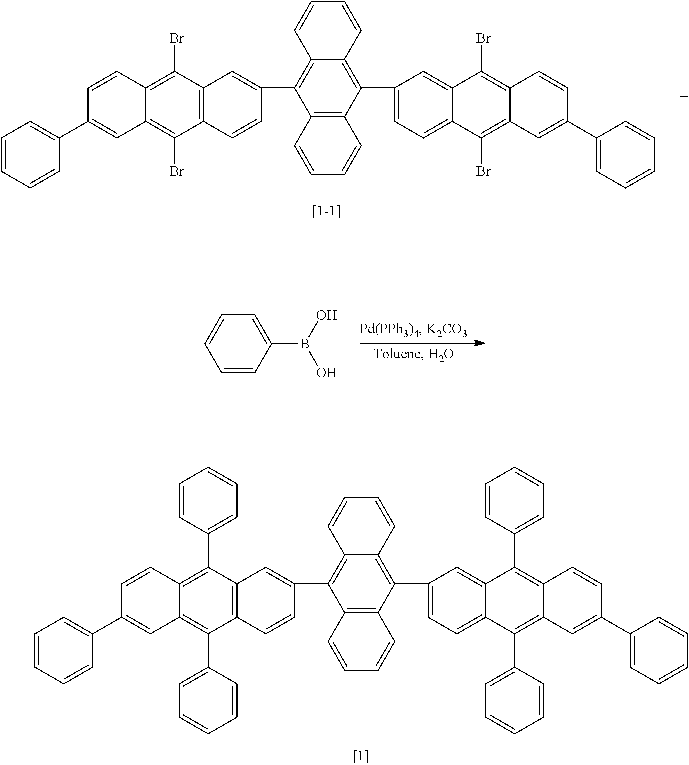

1. Compound [1-1] (49.72 g, 0.05 mol), phenylboronic acid (27.43 g, 0.225 mol), K₂CO₃ (34.55 g, 0.25 mol) and 500 mL of toluene were added into a reaction kettle having a volume of 2 L under a protection of N₂ atmosphere, and stirred.

2. After a temperature of the reaction kettle was increased to 70° C., a catalyst of Pd(PPh₃)₄ (1.16 g, 0.001 mol) and 75 mL of distilled water were added thereinto and stirred for 11 hours. After reacted sufficiently, 100 mL of distilled water was added therein to stop the reaction.

3. A raw product of the target product was obtained after filtered under a vacuum atmosphere, which was washed by distilled water for three times, and then recrystallized using acetone, methylbenzene and THF to obtain a solid, and obtained solid was sublimated for refinement. Lastly, after recrystallizated by methylbenzene, and 36.53 g of the target compound [1] was obtained, being as a light yellow solid with a yield of 74%.

A method for synthesizing the compound represented by formula 8:

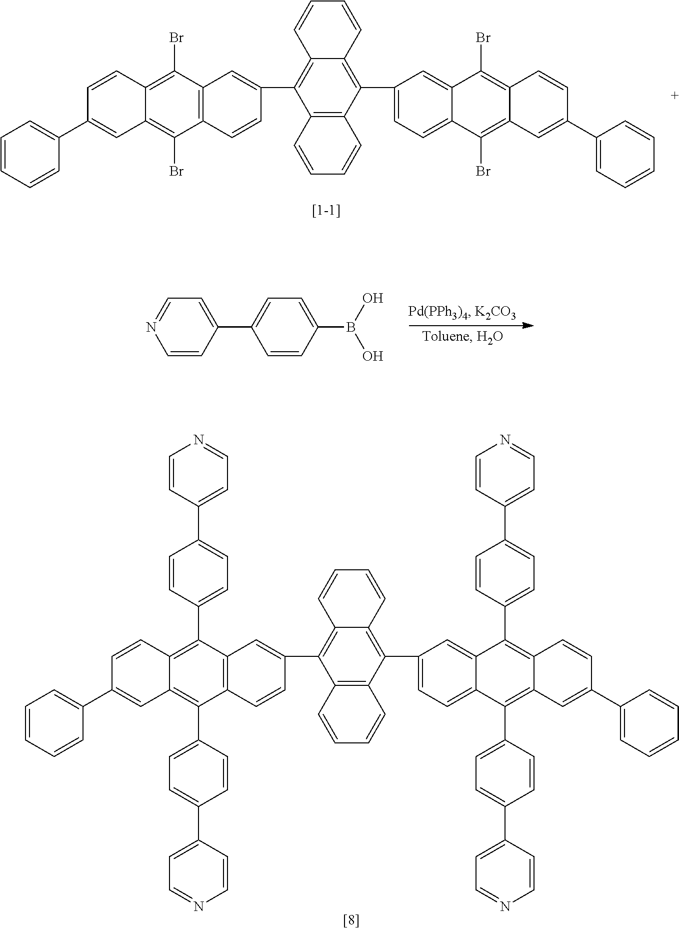 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12 ; OB(O)c1ccc(-c2ccncc2)cc1 ; c1ccc(-c2ccc3c(-c4ccc(-c5ccncc5)cc4)c4cc(-c5c6ccccc6c(-c6ccc7c(-c8ccc(-c9ccncc9)cc8)c8cc(-c9ccccc9)ccc8c(-c8ccc(-c9ccncc9)cc8)c7c6)c6ccccc56)ccc4c(-c4ccc(-c5ccncc5)cc4)c3c2)cc1]

1. Compound [1-1] (49.72 g, 0.05 mol), 4-(4-pyridyl)phenylboronic acid (44.78 g, 0.225 mol), K₂CO₃ (34.55 g, 0.25 mol) and 500 mL of toluene were added into a reaction kettle having a volume of 2 L under a protection of N₂ atmosphere, and stirred.

2. After a temperature of the reaction kettle was increased to 70° C., a catalyst of Pd(PPh₃)₄ (1.16 g, 0.001 mol) and 75 mL of distilled water were added thereinto and stirred for 11 hours. After reacted sufficiently, 100 mL of distilled water was added therein to stop the reaction.

3. A raw product of the target product was obtained after filtered under a vacuum atmosphere, which was washed by distilled water for three times, and then recrystallized using acetone, methylbenzene and THF to obtain a solid, and obtained solid was sublimated for refinement. Lastly, after recrystallizated by methylbenzene, and 50.52 g of the target compound [8] was obtained, being as a light yellow solid with a yield of 78%.

A method for synthesizing the compound represented by formula 11:

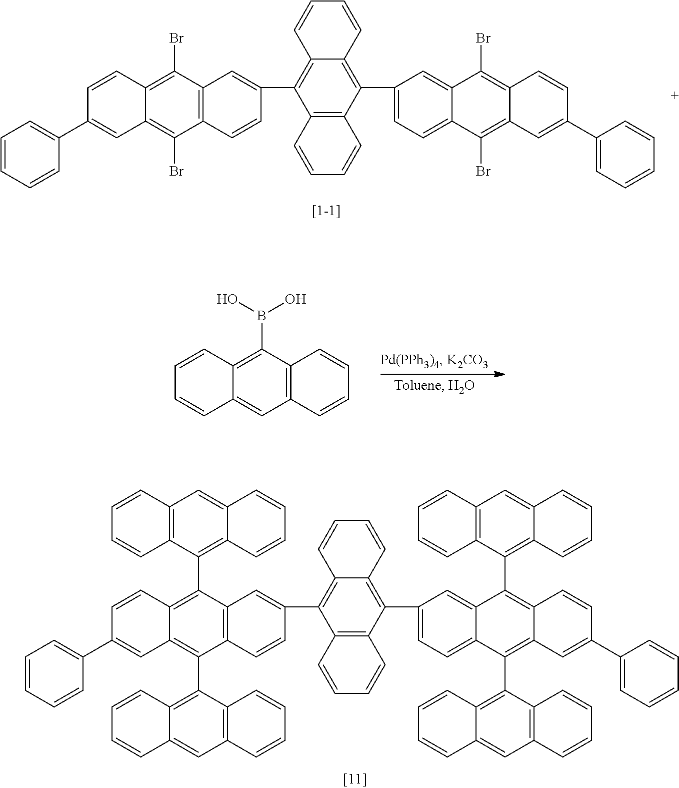

1. Compound [1-1] (49.72 g, 0.05 mol), 9-anthrylboric acid (49.96 g, 0.225 mol), K₂CO₃ (34.55 g, 0.25 mol) and 500 mL of toluene were added into a reaction kettle having a volume of 2 L under a protection of N₂ atmosphere, and stirred.

2. After a temperature of the reaction kettle was increased to 70° C., a catalyst of Pd(PPh₃)₄ (1.16 g, 0.001 mol) and 75 mL of distilled water were added thereinto and stirred for 11 hours. After reacted sufficiently, 100 mL of distilled water was added to stop the reaction.

3. A raw product of the target product was obtained after filtered under a vacuum atmosphere, which was washed by distilled water for three times, and then recrystallized using acetone, methylbenzene and THF to obtain a solid, and obtained solid was sublimated for refinement. Lastly, after recrystallizated by methylbenzene, 52.71 g of the target compound [11] was obtained, being as a light yellow solid with a yield of 76%.

Process for synthesizing the anthracene compounds represented by formulas 1 to 12 in Table 1 is general same, which are not detailed described herein for brevity, with specific results thereof shown in Table 2. Besides, during the process for preparing the anthracene compound according to the present disclosure, an amount of each added material can be determined based on a proportion of the added materials in the above example. The focus of the present disclosure is to describe the process for preparing the anthracene compound; the amount of each added material is not detailed described herein.

TABLE 2

| | | MS/FAB |
|---|---|---|
| No. | Elemental analysis | (M⁺) |
| 1 | calculating value -- C: 94.90%; H: 5.10%; | 987.23 |
| | testing value -- C: 94.92%; H: 5.08%; | |
| 2 | calculating value -- C: 94.85%; H: 5.15%; | 1291.62 |
| | testing value -- C: 94.84%; H: 5.16%; | |
| 3 | calculating value -- C: 95.08%; H: 4.92%; | 1187.47 |
| | testing value -- C: 95.09%; H: 4.91%; | |
| 4 | calculating value -- C: 90.73%; H: 4.57%; N: 4.70%; | 1191.42 |
| | testing value -- C: 90.73%; H: 4.56%; N: 4.71%; | |
| 5 | calculating value -- C: 95.21%; H: 4.79%; | 1387.70 |
| | testing value -- C: 95.21%; H: 4.79%; | |
| 6 | calculating value -- C: 89.67%; H: 4.68%; N: 5.65%; | 991.18 |
| | testing value -- C: 89.64%; H: 4.69%; N: 5.67%; | |
| 7 | calculating value -- C: 95.03%; H: 4.97%; | 1339.66 |
| | testing value -- C: 95.03%; H: 4.97%; | |
| 8 | calculating value -- C: 90.85%; H: 4.82%; N: 4.33%; | 1295.57 |
| | testing value -- C: 90.84%; H: 4.81%; N: 4.35%; | |
| 9 | calculating value -- C: 94.31%; H: 5.69%; | 1451.87 |
| | testing value -- C: 94.31%; H: 5.69%; | |
| 10 | calculating value -- C: 94.82%; H: 5.18%; | 1596.00 |
| | testing value -- C: 94.81%; H: 5.19%; | |
| 11 | calculating value -- C: 94.31%; H: 5.69%; | 1387.70 |
| | testing value -- C: 94.32%; H: 5.68%; | |
| 12 | calculating value -- C: 81.03%; H: 3.81%; N: 4.61%; | 1215.53 |
| | S: 10.55%; | |
| | testing value -- C: 81.01%; H: 3.82%; N: 4.63%; | |
| | S: 10.54%; | |

In the following description, the anthracene compound according to the present disclosure is described in further details by being used as a fluorescent green host material.

### Comparative Example 1

Hereinafter, an organic light emitting device with following structure was manufactured as the comparative sample 1 by using compound a as a fluorescent green host material, using compound c as a fluorescent green dopped material, using 2-TNATA as a hole injection layer material, using α-NPD (N,N′-dinaphthyl-N,N′-diphenybenzidine) as a hole transport layer material. The organic light emitting device had a structure of ITO/2-TNATA (80 nm)/α-NPD (30 nm)/compound a+compound c (30 nm)/Alq₃ (30 nm)/LiF (0.5 nm)/Al (60 nm).

The anode was made from 15 Ω/cm² (1000 Δ) of an ITO glass substrate, which was cut into a size of 50 mm*50 mm*0.7 mm, and washed under ultrasonic wave using acetone, isopropanol, purified water for 15 min respectively, and then washed in UV-ozone for another 30 min. A layer of 2-TNATA having a thickness of 80 nm was deposited on obtained substrate by a vacuum evaporation to form a hole injection layer. And a layer of α-NPD having a thickness of 30 nm was deposited on the hole injection layer by a vacuum evaporation to form a hole transport layer. Then a layer of compound a and compound c (3% coating) was deposited on the hole transport layer by a vacuum evaporation to a light emitting layer having a thickness of 30 nm. And then, a layer of Alq₃ having a thickness of 30 nm was deposited on the light emitting layer by a vacuum evaporation to form an electron transport layer. Lastly, a layer of LiF having a thickness of 0.5 nm (electron injection layer) and a layer of Al having a thickness of 60 nm (cathode) were successively deposited on the electron transport layer respectively.

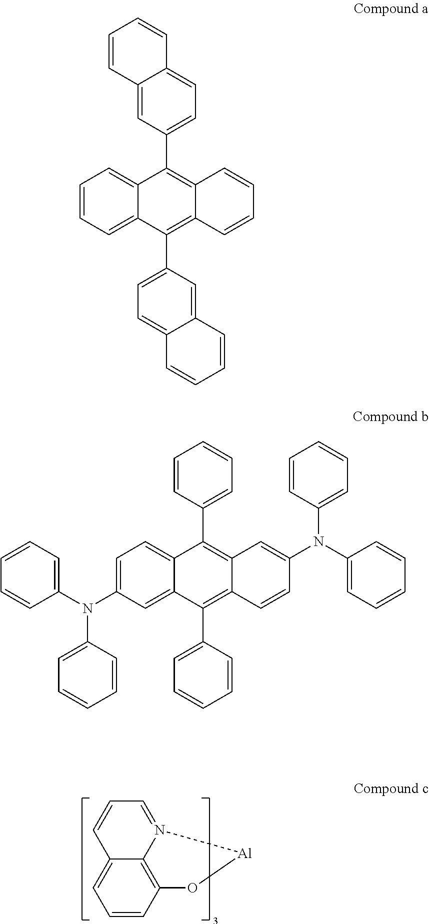

### Examples 1 to 12

Organic electroluminescent devices 1 to 12 were manufactured by a method and a condition being same as that in the comparative example 1, except the compound a being as the fluorescent host material was replaced by the anthracene compound represented by formulas 1 to 12 in Table 1 respectively.

The light emitting properties of the comparative sample 1 and the samples 1 to 12 were measured. A brightness, a light emitting efficiency, a light color were evaluated using a measuring unit Keithley SMU235. The same tests were carried out with the comparative sample 1 and the samples 1 to 12, and obtained results were shown in Table 3.

TABLE 3

| | Host | Dopped | Bright- | Light emitting | Wave- |
|---|---|---|---|---|---|
| | com- | com- | ness | efficiency | length |
| No. | pound | pound | [cd/m²] | [cd/A] | [nm] |
| Comparative | a | b | 2032 | 20.3 | 516 |
| Sample | | | | | |
| Sample 1 | 1 | b | 2538 | 25.4 | 516 |
| Sample 2 | 2 | b | 3140 | 31.4 | 519 |
| Sample 3 | 3 | b | 3029 | 29.3 | 521 |
| Sample 4 | 4 | b | 2942 | 29.4 | 518 |
| Sample 5 | 5 | b | 3001 | 21.4 | 523 |
| Sample 6 | 6 | b | 3155 | 23.6 | 520 |
| Sample 7 | 7 | b | 2736 | 27.4 | 512 |
| Sample 8 | 8 | b | 2828 | 28.3 | 518 |
| Sample 9 | 9 | b | 2756 | 26.6 | 521 |
| Sample 10 | 10 | b | 2967 | 27.7 | 519 |
| Sample 11 | 11 | b | 2934 | 28.3 | 512 |
| Sample 12 | 12 | b | 2595 | 26.0 | 524 |

As shown in Table 3, all samples show a color of emitting lights being as green within a wavelength ranging from 512 nm to 524 nm. The light emitting efficiency and the brightness of the samples 1 to 12 are significantly improved comparing with the comparative sample 1.

Although explanatory embodiments have been described and shown, it would be appreciated by those skilled in the art that various changes in forms and details can be made in the embodiments without departing from the spirit and scope of the present disclosure defined by following claims.

The above are merely the preferred embodiments of the present disclosure. It should be noted that, a person skilled in the art may further make improvements and modifications without departing from the principle of the present invention, and these improvements and modifications shall also be considered as the scope of the present disclosure.

What is claimed is:

1. An anthracene compound, represented by a formula: 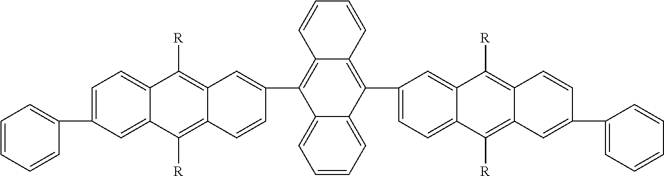
wherein R is selected from C6-C19 aryl, fused ring aryl, or substituted or unsubstituted heterocyclic aryl.

2. The anthracene compound according to claim 1, wherein R is selected from C6-C50 phenyl, biphenyl, naphthyl, quinolyl, phenanthryl, pyridyl, phenalenyl, 9,9-dimethyl-fluorenyl, terphenyl, anthryl, aromatic azyl, carbazolyl, benzothiazolyl, thienyl, substituted or unsubstituted heterocyclic aryl, or anilino.

3. The anthracene compound according to claim 1, wherein the anthracene compound is selected from any one of followings:

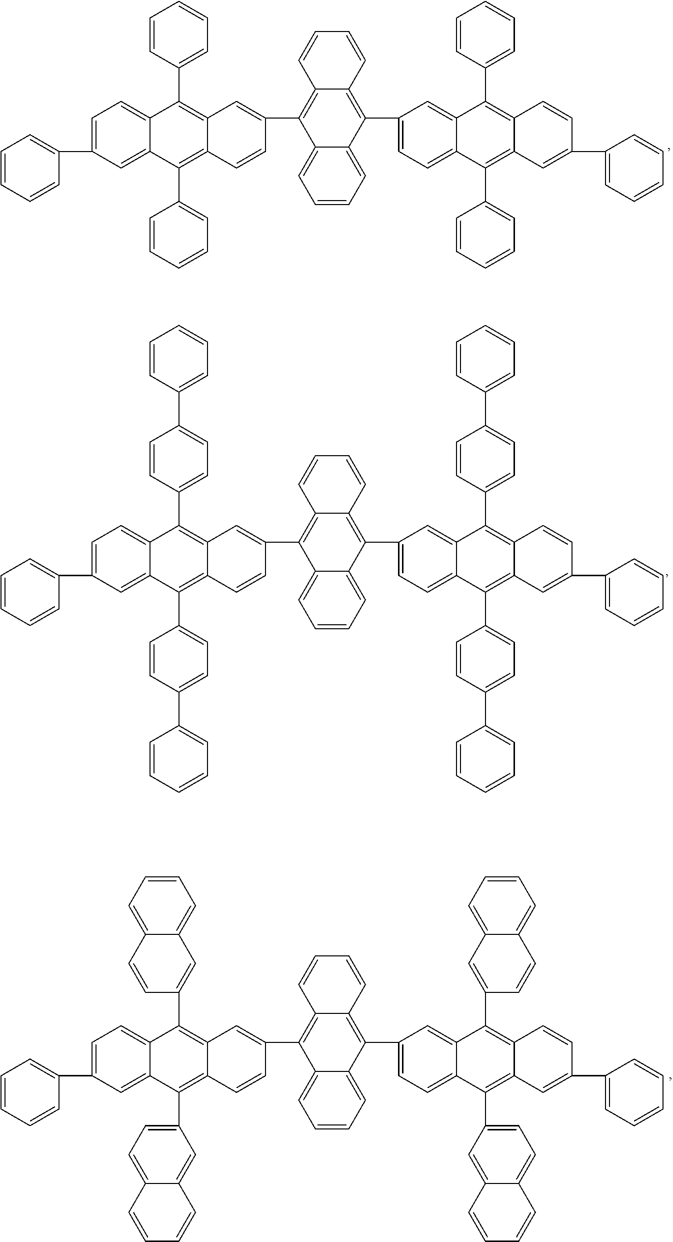

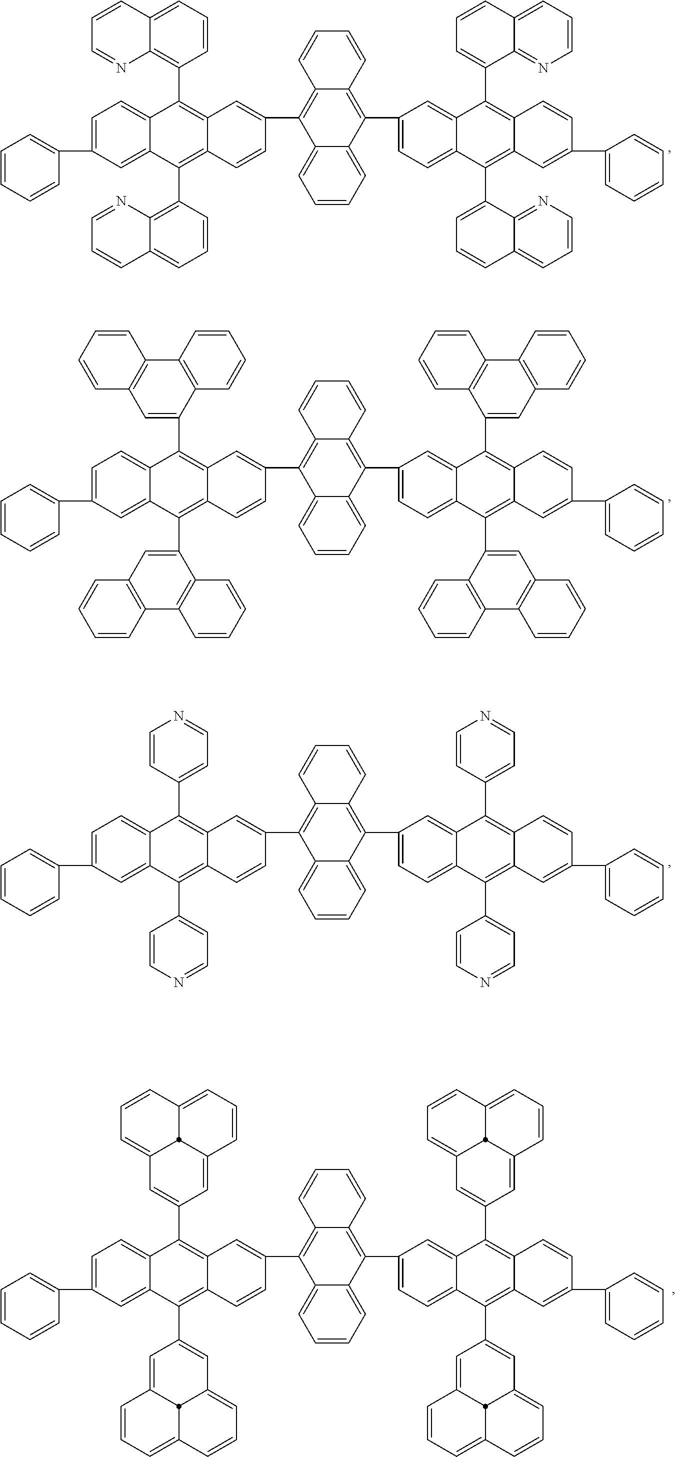

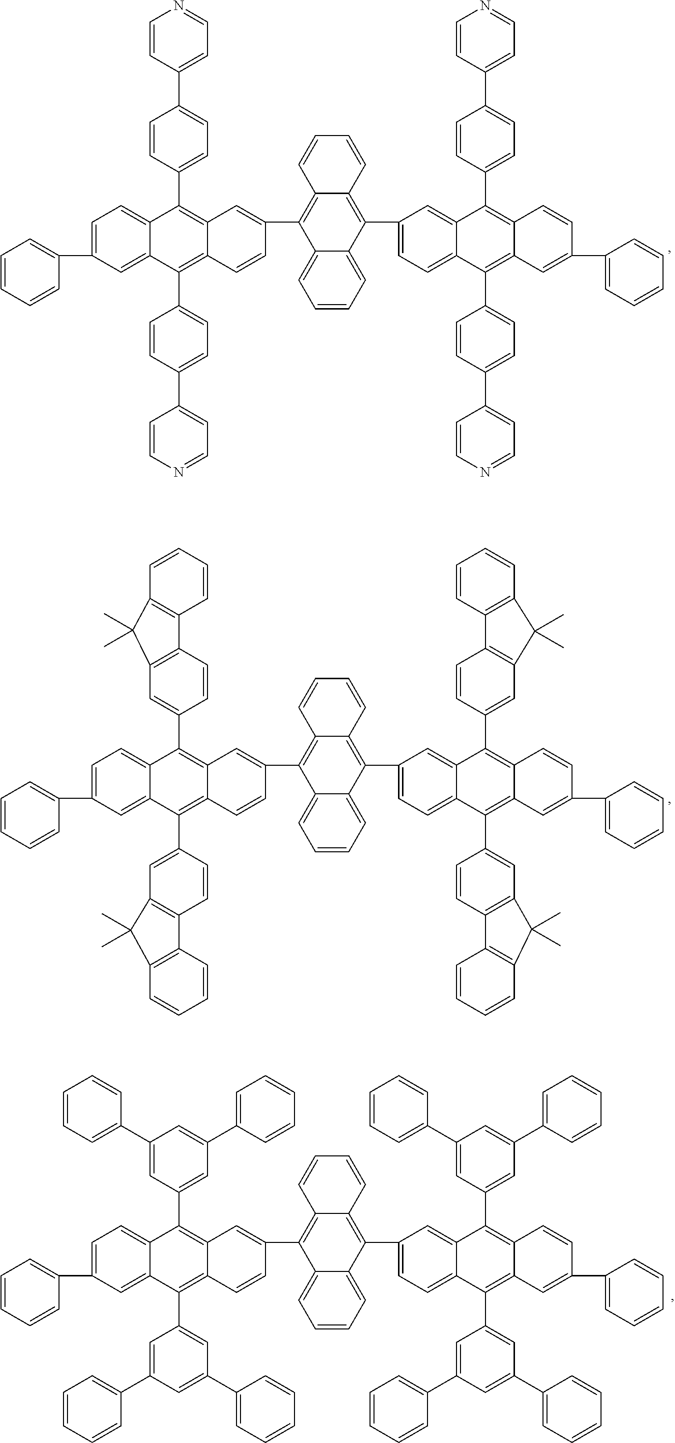

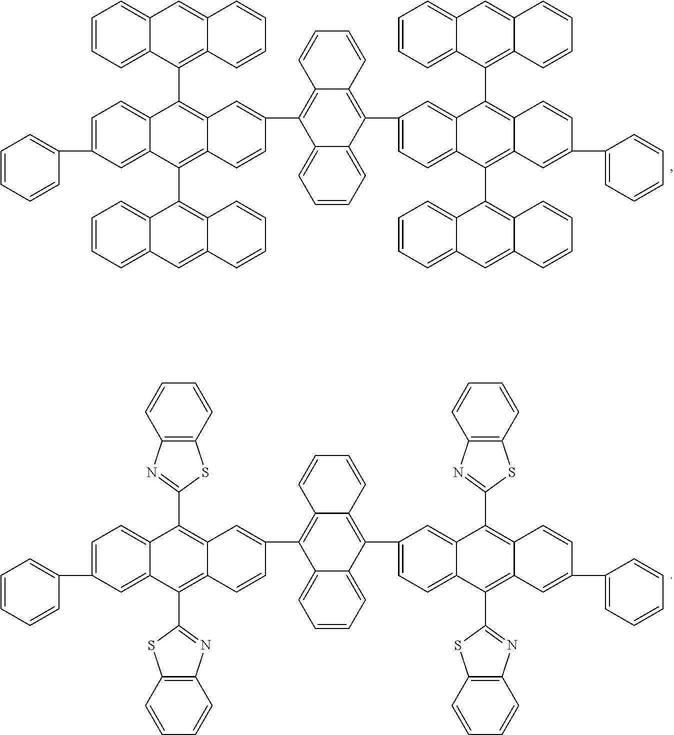

4. A method for utilizing the anthracene compound according to claim 1 in an organic electroluminescent device, comprising: using the anthracene compound as a fluoresce host material, a hole injection material or a hole transport material in the organic electroluminescent device.

5. The method according to claim 4, wherein the anthracene compound is used as a fluorescent green host material in the organic electroluminescent device.

6. An organic electroluminescent device, comprising:
a first electrode;
a second electrode; and
one or more organic compound layers between the first electrode and the second electrode,
wherein at least one organic compound layer comprises the anthracene compound according to claim 1.

7. A method for preparing the anthracene compound according to claim 1, comprising the following steps:
step S1: degassing a reaction vessel, and adding 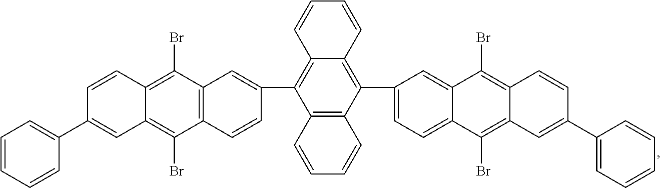 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]
R-boric acid, potassium carbonate and methylbenzene thereinto;
step S2: adding a catalyst, increasing a temperature of the reaction vessel to 70° C. and refluxing for reacting sufficiently; and
step S3: extracting, washing, drying and purifying by column chromatography, to obtain the anthracene compound,
wherein R is selected from C6-C19 aryl, fused ring aryl, or substituted or unsubstituted heterocyclic aryl.

8. The method according to claim 7, wherein in the step S1, 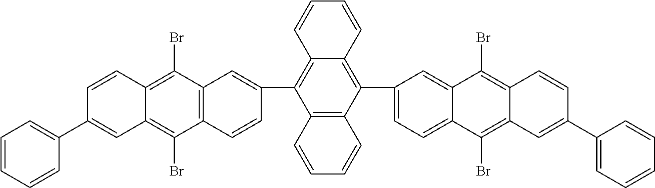 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]
is obtained from 9,10-anthraquinone.

9. The method according to claim 7, wherein in the step S1, 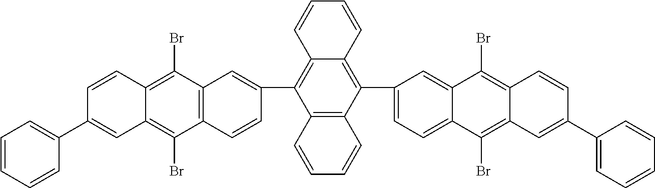 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]
is obtained by a multi-step reaction of alcoholization, dehydration and bromination using 2-bromo-6-benzanthracene and 9,10-anthraquinone as raw materials, the step S1 further comprises the following steps:
step N1: degassing a reaction vessel, and adding 2-bromo-6-benzanthracene and tetrahydrofuran thereinto;
step N2: decreasing a temperature of a reaction system, and adding n-BuLi;
step N3: adding 9,10-anthraquinone;
step N4: increasing the temperature of the reaction system to a room temperature, and adding NH₄Cl to stop the reaction after reacting sufficiently;
step N5: extracting, washing, drying and purifying by column chromatography to obtain 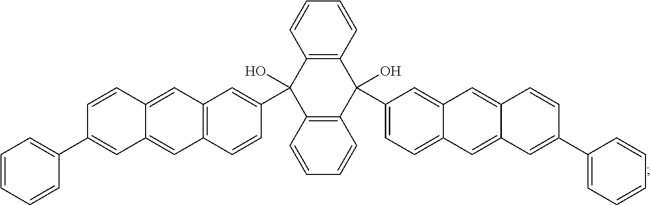 [SMILES: OC1(c2ccc3cc4cc(-c5ccccc5)ccc4cc3c2)c2ccccc2C(O)(c2ccc3cc4cc(-c5ccccc5)ccc4cc3c2)c2ccccc21]
step N6: adding potassium iodide, sodium dihydrogen phosphate and acetic acid to obtain 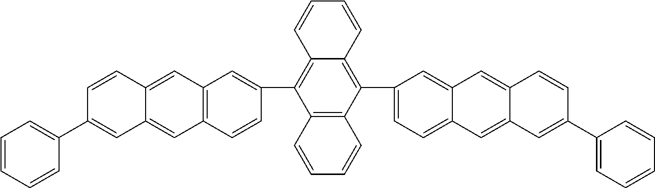 [SMILES: c1ccc(-c2ccc3cc4cc(-c5c6ccccc6c(-c6ccc7cc8cc(-c9ccccc9)ccc8cc7c6)c6ccccc56)ccc4cc3c2)cc1]
by a dehydration reaction; and
step N7: brominating by adding bromine water to obtain 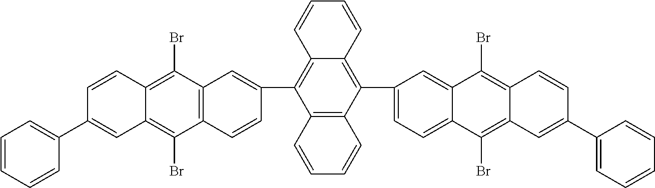 [SMILES: Brc1c2ccc(-c3c4ccccc4c(-c4ccc5c(Br)c6cc(-c7ccccc7)ccc6c(Br)c5c4)c4ccccc34)cc2c(Br)c2ccc(-c3ccccc3)cc12]
in the step S1.

10. The method according to claim 7, wherein R-boric acid is selected from phenylboric acid, 4-biphenylboric acid, 2-naphthylboric acid, 8-quinolylboric acid, 9-phenanthrylboric acid, 4-pyridylboric acid, phenalenylboric acid, 4-(4-pyridyl)-phenylboric acid, 9,9-dimethyl-fluorenylboric acid, 3,5-diphenyl-phenylboric acid, 9-anthrylboric acid, or 2-benzothiazolylboric acid.

11. An organic electroluminescent device, comprising:
a first electrode;
a second electrode; and
one or more organic compound layers between the first electrode and the second electrode,
wherein at least one organic compound layer comprises the anthracene compound according to claim 2.

12. An organic electroluminescent device, comprising:
a first electrode;
a second electrode; and
one or more organic compound layers between the first electrode and the second electrode,
wherein at least one organic compound layer comprises the anthracene compound according to claim 3.
